(19) United States
(12) Patent Application Publication
(10) Pub. No.: US 2010/0055195 A1
(43) Pub. Date: Mar. 4, 2010

(54) BIODEGRADABLE BMP NANOFIBER AND USES THEREOF

(75) Inventor: Seung Bum PARK, Gwanak-Gu (KR)

Correspondence Address: Seung Bum Park;122-I-502 Seoul National University, Faculty Apartment, Bongchun-dong, Gwanak-Gu, Seoul, KR

(73) Assignee: SEOUL NATIONAL UNIVERSITY

(21) Appl. No.: 12/201,885

(22) Filed: Aug. 29, 2008

(51) Int. Cl. A61K 9/14; A61K 38/16

(52) U.S. Cl. 424/499; 514/12

(57) ABSTRACT

Described herein are compositions and methods for treating bone disorders. The compositions and methods relate to a nanofiber comprising one or more of one or more bone morphogenetic proteins, or one or more bone morphogenetic protein fragments bound to the nanofiber.

### BACKGROUND

Bone Morphogenetic Proteins (BMPs) are a group of growth factors and cytokines known for their ability to induce the formation of bone and cartilage. Originally, seven BMPs proteins were discovered. Of these, six of them (BMP2 through BMP7) belong to the transforming growth factor beta superfamily of proteins. Since then, thirteen more BMPs have been discovered, bringing the total to twenty.

BMPs interact with specific receptors on the cell surface, referred to as bone morphogenetic protein receptors (BMPRs). Signal transduction through BMPRs results in mobilization of members of the SMAD family of proteins. The signaling pathways involving BMPs, BMPRs and SMADs are important in the development of the heart, central nervous system, and cartilage, as well as post-natal bone development.

As inefficient methods are used to deliver BMPs for the treatment of bone disorders, there is a need for more efficient and directed methods and compositions for BMP therapy.

### SUMMARY

Embodiments relating to compositions and methods for treating a bone disorder in a subject are provided. The compositions relate to the unexpected finding that bone morphogenetic proteins (BMPs) can be bound to a nanofiber and administered to a subject at the site of a bone disorder. The administration of the nanofiber is can be directed and local, and the administration of the BMP can be through release or contact, such that a therapeutically effective amount of the BMP is administered.

One embodiment is directed to a nanofiber comprising: one or more of one or more BMPs, or one or more BMP fragments bound to the nanofiber. In some embodiments, the nanofiber is selected from the group consisting of: a carbon nanofiber, a polymeric nanofiber (e.g., nylon, polystyrene, polyacrylonitrile, polycarbonate, poly(ethylene oxide), polyethylene terephthalate, and water soluble polymers), an organic crystalline nanofiber, an inorganic phosphate nanofiber, a co-polymeric nanofiber, and a core-shell type nanofiber. In some embodiments, the one or more of the one or more BMPs, or the one or more BMP fragments, is selected from the group consisting of: BMP2; BMP3; BMP4; BMP5; BMP6; BMP7, and one or more fragments thereof. In some embodiments, the one or more of the one or more BMPs, or the one or more BMP fragments, comprises a polypeptide with at least about 85% identity to SEQ ID NOS: 1-7 or a functional fragment thereof.

In some embodiments, the one or more of the one or more BMPs, or the one or more BMP fragments, is attached to the nanofiber by a linker molecule (e.g., N-succinimidyl-3(2 pyridyldithio)-propionate, amine-containing cross-linker (SMCC), bis-maleimidohexane, dimethyl pimelimidate, dithiobis-(succinimidyl propionate), disuccinimidyl suberate, and related linkers). In some embodiments, the one or more of the one or more BMPs, or the one or more BMP fragments, is coupled to the nanofiber with a linkage comprising a dithio bond. In some embodiments, the linker comprises a hydrolysable functional group. In some embodiments, the nanofiber is biodegradable. In some embodiments, the nanofiber is poly(D,L-lactic lycine). In some embodiments, the nanofiber further comprises a pharmaceutically acceptable carrier.

One embodiment, is directed to a method of treating a bone disorder or disease in a subject, comprising: administering an effective amount of a nanofiber comprising one or more of one or more BMPs, or one or more BMP fragments bound to the nanofiber, wherein administration of the nanofiber results in the treatment of the bone disorder or disease. In some embodiments, the bone disorder or disease is selected from the group consisting of: a fracture, a microfracture and osteoporosis. In some embodiments, the treatment of the subject is achieved by an increase in the bone density of the subject.

One embodiment is directed to a method of increasing bone density in a subject, comprising: administering an effective amount of a nanofiber comprising one or more of one or more BMPs, or one or more BMP fragments bound to the nanofiber, wherein administration of the nanofiber results in an increase in the bone density of the subject. In some embodiments, the one or more of the one or more BMPs, or the one or more BMP fragments, is administered by local release from the nanofiber in a controlled manner. In some embodiments, the local release of the one or more of the one or more BMPs, or the one or more BMP fragments is accomplished by hydrolysis of a hydrolysable functional group, e.g., a phenolic ester or thioester, in a linker attaching the one or more of the one or more BMPs, or the one or more BMP fragments to the nanofiber.

One embodiment is directed to a method of activating MAPK extracellular regulated kinase pathway, comprising: administering an effective amount of a nanofiber comprising one or more of one or more BMPs, or one or more BMP fragments bound to the nanofiber, wherein administration of the nanofiber activates the MAPK regulated kinase pathway.

One embodiment is directed to a method of inducing phosphorylation of SMAD1 or SMAD5 at the site of a bone disorder or disease in a subject, comprising: administering an effective amount of a nanofiber comprising one or more of one or more BMPs, or one or more BMP fragments bound to the nanofiber at the site of the bone disorder or disease, wherein administration of the nanofiber results in phosphorylation of SMAD1 or SMAD5 at the site of the bone disorder in the subject.

One embodiment is directed to a kit comprising: a) a nanofiber as described herein; b) a solution suitable for storing the protein nanofiber of a); and c) instructions for the use of the nanofiber.

One embodiment is directed to the use of a nanofiber comprising one or more of one or more BMPs, or one or more BMP fragments bound to the nanofiber, for treating a bone disorder or disease. In some embodiments, the bone disorder or disease is selected from the group consisting of: a fracture; a microfracture; and osteoporosis.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1A-B show the amino acid sequences of BMP2 (SEQ ID NO:1), BMP3 (SEQ ID NO:2), BMP4 (SEQ ID NO:3), BMP5 (SEQ ID NO:4), BMP6 (SEQ ID NO:5), BMP7 (SEQ ID NO:6 and BMP8 (SEQ ID NO:7).

FIG. 2 is a conjugation reaction scheme showing an illustrative embodiment of the attachment of a BMP to a nanofiber using a linker.

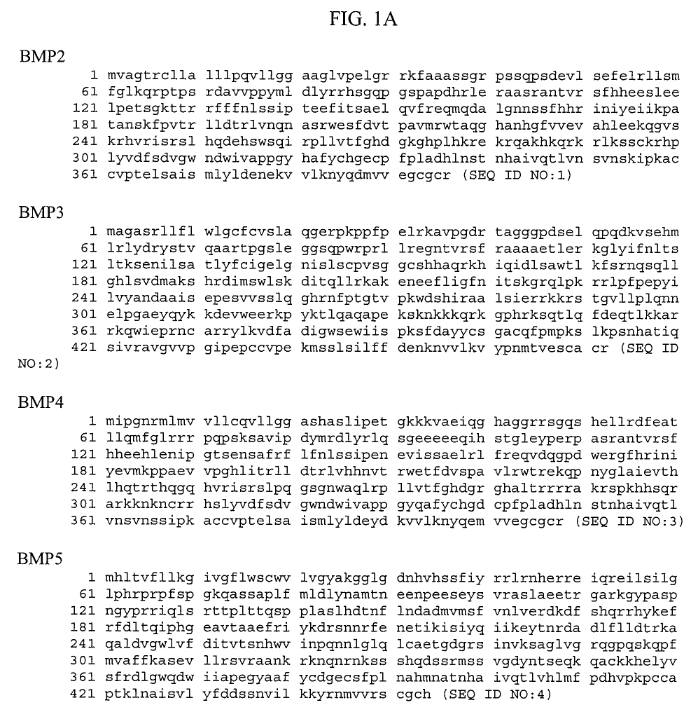

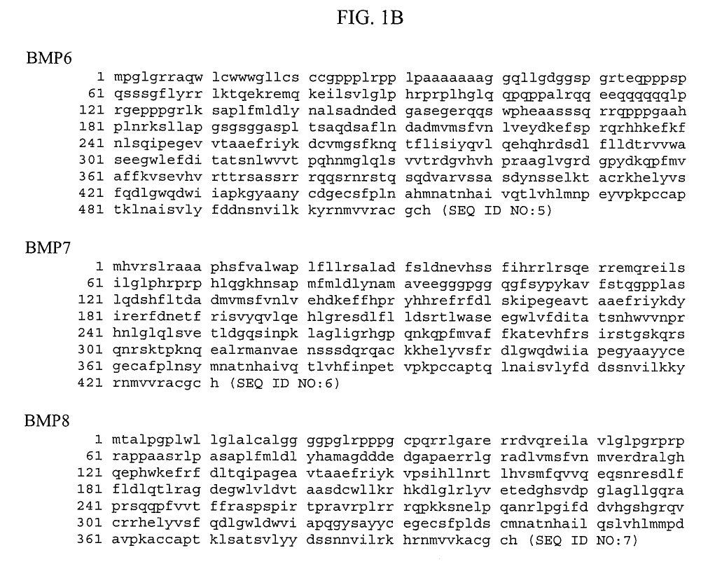

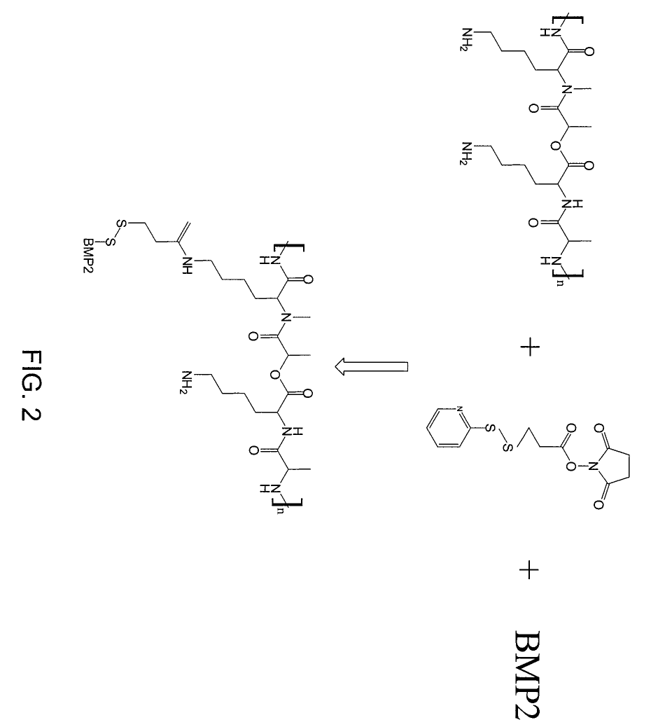

### DETAILED DESCRIPTION

In the following detailed description, reference is made to the accompanying drawing, which form a part hereof. The illustrative embodiments described in the detailed description, drawing, and claims are not meant to be limiting. Other embodiments may be utilized, and other changes may be made, without departing from the spirit or scope of the subject matter presented here.

Described herein are compositions and methods for treating bone disorders, e.g., a fracture, microfracture or osteoporosis. Compositions are directed to nanofibers that have one or more bone morphogenetic proteins (BMPs) bound. Administration of the BMP-bound nanofiber to a subject at the site of a bone disorder, for example, allows for the directed and local delivery of a therapeutically effective amount of the BMP. In certain embodiments, the BMP is bound to the nanofiber via a linker. In certain embodiments, the linker can comprise a hydrolysable functional group that allows for specific release of the BMP at the site of a bone disorder after hydrolysis of the hydrolysable functional group.

“Nanofibers” are structural fibers that have a submicron dimension. The dimension can be measured across the largest portion of the particle. The dimension can be a length, width or diameter of the particle. Nanofibers include, for example, carbon nanofiber, polymeric nanofiber (e.g., nylon, polystyrene, polyacrylonitrile, polycarbonate, poly(ethylene oxide) (PEO), polyethylene terephthalate (PET), and water soluble polymers), organic crystalline nanofiber, inorganic phosphate nanofibers, co-polymeric nanofibers, and core-shell type nanofibers. Nanofibers can be manipulated to form structures, or they can self-assemble into structures that allow for administration of the nanofiber to a particular site of injury in a subject, e.g., the site of a bone disorder.

Bone morphogenetic proteins have the ability to modulate bone formation through, for example, osteoblast differentiation leading to increased bone density. They are a group of factors that can modulate bone structure and repair, i.e., they can stimulate bone formation (and thereby increase bone density) or bone resorption. Specific BMPs Include, for example, BMP2, BMP3, BMP4, BMP5, BMP6, BMP7 and BMP8.

BMP2 induces bone and cartridge formation and plays a key role in osteoblast differentiation. BMP2 acts as a disulfide-linked homodimer and induces bone and cartilage formation. It is a candidate as a retinoid mediator. BMP3 induces bone formation. BMP4 regulates the formation of teeth, limbs, and bone from mesoderm. It also plays a role in fracture repair. BMP5 induces cartridge formation. BMP6 plays a role in joint integrity in adults. BMP7 plays a key role in osteoblast differentiation and induces the production of SMAD1. BMP8 Involved in bone and cartilage development.

The cytokines LIF and BMP2 signal through different receptors and transcription factors, namely STATs and SMADs, respectively. LIF and BMP2 act in synergy on primary fetal neural progenitor cells to induce astrocytes (Nakashima, K. et al., Science, 284:479-482, 1999.). The formation of a complex between STAT3 and SMAD1, bridged by p300, is involved in the cooperative signaling of LIF and BMP2 and the subsequent induction of astrocytes from neuronal progenitors.

The human SMAD1 gene encodes a 465-amino acid polypeptide that is 76% identical to Drosophila Mad and 42% identical to human DPC4 (Liu, F. et al., Nature, 381:620-623, 1996). The human gene product is phosphorylated and localizes to the nucleus upon activation by bone morphogenetic protein (BMP) subfamily members (e.g., BMP2).

SMAD5 plays a critical role in the signaling pathway by which TGF-beta inhibits the proliferation of human hematopoietic progenitor cells (Bruno, E. et al., Blood, 91:1917-1923, 1998). SMAD5 gene has 8 exons, with the coding sequence contained in exons 3 to 8 (Gemma, A. et al., Oncogene, 16:951-956, 1998). The SMAD5 protein has strong homology with SMAD1, SMAD2, SMAD3 and SMAD4 in the N- and C-terminal domains, which are separated by a proline-rich sequence. SMAD5 shows the greatest homology to SMAD1.

The BMPs described herein, whether signaling through SMAD proteins or other signaling pathways, are polypeptides that can be bound to nanofibers for directed and local administration, which includes local release of the BMPs from the nanofiber. The term “polypeptide” refers to a polymer of amino acids, and not to a specific length or state of post-translational modification; peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell and still be “isolated” or “purified.” A polypeptide can be purified to homogeneity. It is understood, however, that preparations in which the polypeptide is not purified to homogeneity are useful. The critical feature is that the preparation allows for the desired function of the polypeptide, even in the presence of considerable amounts of other components. In one embodiment, the language “substantially free of cellular material” includes preparations of the polypeptide having less than about 30% (by dry weight) other proteins (e.g., contaminating protein), less than about 20% other proteins, less than about 10% other proteins, or less than about 5% other proteins.

As used herein the terms “Bone morphogenetic Protein” or “BMP polypeptide” refer to a polypeptide sequence of a BMP or a polypeptide sequence with at least about 70-75% sequence identity to the polypeptide sequence of a BMP. As used herein, two polypeptides (or a region of the polypeptides) are substantially identical when the amino acid sequences are at least about 70-75% identical. The BMP polypeptide useful for the methods herein can be, for example, about 70% identical to a BMP sequence, about 75% identical to a BMP sequence, about 80% identical to a BMP sequence, about 85% identical to a BMP sequence, about 90% identical to a BMP sequence, or about 95% identical to a BMP sequence. An amino acid sequence substantially identical to a BMP sequence will be encoded by a nucleic acid molecule hybridizing to a nucleic acid sequence that encodes a BMP sequence, or portion thereof, under stringent conditions as determined by those of skill in the art.

To determine the percent identity of two amino acid sequences, the sequences are aligned for optimal comparison purposes (e.g., gaps can be introduced in the sequence of one polypeptide sequence for optimal alignment with the other polypeptide sequence). The amino acid residues at corresponding amino acid positions or nucleotide positions are then compared. Where a position in one sequence is occupied by the same amino acid residue or nucleotide as the corresponding position in the other sequence, then the molecules are homologous at that position. The percent identity between the two sequences is a function of the number of identical positions shared by the sequences (i.e., percent identity equals the number of identical positions/total number of positions times 100).

Useful for the methods herein are polypeptides having a lower degree of identity but having sufficient similarity so as to perform one or more of the same functions performed by a BMP polypeptide. Similarity is determined by conserved amino acid substitution. Such substitutions are those that substitute a given amino acid in a polypeptide by another amino acid of like characteristics. Conservative substitutions are likely to be phenotypically silent. Conservative substitutions, for example, are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gin, exchange of the basic residues Lys and Arg and replacements among the aromatic residues Phe and Tyr. Guidance concerning which amino acid changes are likely to be phenotypically silent is found in, for example, Bowie, J. et al., Science, 247:1306-1310, 1990.

A “fragment” refers to molecule that has only a portion of a full-length sequence. A BMP polypeptide fragment, for example, is a truncated BMP. Fragments can contain sequence from either end of the full-length sequence, or they can contain a sequence from the middle of a full-length sequence. A fragment can be a “functional fragment,” e.g., a fragment that retains one or more functions of the full-length polypeptide, or a fragment can be a “non-functional fragment,” e.g., a fragment that does not retain a specified activity of the full-length polypeptide. Fragments of full-length variant polypeptides are also useful for the compositions and methods described herein.

A “variant” polypeptide can differ in amino acid sequence by one or more substitutions, deletions, insertions, inversions, fusions, and truncations or a combination of any of these. Variant polypeptides can be fully functional or can lack function in one or more activities. Fully functional variants can contain, for example, only conservative variation or variation in non-critical residues or in non-critical regions. Functional variants can also contain substitution of similar amino acids that result in no change or an insignificant change in function. Alternatively, such substitutions can positively or negatively affect function to some degree. Non-functional variants commonly contain one or more non-conservative amino acid substitutions, deletions, insertions, inversions, or truncation or a substitution, insertion, inversion, or deletion in a critical residue or critical region. Amino acids that are essential for function can be identified by methods known in the art, such as, for example, site-directed mutagenesis or alanine-scanning mutagenesis (Cunningham, B. and Wells, J., Science, 244:1081-1085, 1989). Sites that are critical for polypeptide activity can also be determined, for example, by structural analysis such as crystallization, nuclear magnetic resonance or photoaffinity labeling (Smith, L. et al., J. Mol. Biol., 224:899-904, 1992; de Vos, A. et al., Science, 255:306-312, 1992).

BMPs can be bound either directly to a nanofiber or they can be attached to the nanofiber via a linker. The introduction of linkers or spacers between the nanofiber and the BMP polypeptide can allow for optimized administration of the BMP to the subject and/or release of the BMP from the nanofiber. Use of such linkers/spacers, for example, can improve the relevant properties of the BMP polypeptides (e.g., increase serum stability, ease of attachment to the nanofiber, specific release of the BMP for the nanofiber, etc.). Linkers include, for example, N-succinimidyl-3(2 pyridyldithio)-propionate, amine-containing cross-linker (SMCC), bis-maleimidohexane, dimethyl pimelimidate, dithiobis-(succinimidyl propionate), disuccinimidyl suberate, and related cross linkers.

Linkers can also comprise one or more hydrolysable functional groups, e.g., such as phenolic esters or thioesters, or cleavable moieties that allow for specific release of BMPs from the nanofiber in a desired microenvironment. Linkers include, for example, homobifunctional and heterobifunctional cross-linking molecules. The homobifunctional molecules have at least two reactive functional groups that are the same. Heterobifunctional linker molecules have at least two different reactive groups. Some examples of heterobifunctional reagents containing reactive disulfide bonds include N-succinimidyl 3-(2-pyridyl-dithio)propionate, sodium S-4-succinimidyloxycarbonyl-alpha-methylbenzylthiosulfate, and 4-succinimidyloxycarbonyl-alpha-methyl-(2-pyridyldithio)toluene.

Methods for attaching a BMP to a nanofiber include, for example, attachment via a dithio bond—either directly or as contained in a linker. Some examples of heterobifunctional reagents comprising reactive groups having a double bond that reacts with a thiol group include succinimidyl 4-(N-maleimidomethyl)cyclohexane-1-carboxylate and succinimidyl m-maleimidobenzoate.

Linkers that are combinations of the molecules and/or moieties described above, can also be employed to confer special advantage to the properties of the peptide. Such constructs could be employed as agents for targeting and delivery of a diagnostic reporter or a therapeutic agent, or a combination of these.

BMP nanofibers described herein are useful for treating bone disorders, as they allow for directed and/or controlled delivery of BMPs at the site of a bone disease or disorder, e.g., a fracture, a microfracture or osteoporosis, in a subject. As used herein, the term “subject” refers to an animal. The animal can be, for example, a mammal, either human or non-human. A subject can also refer to, for example, primates (e.g., monkeys, apes and humans), cows, pigs, sheep, goats, horses, dogs, cats, rabbits, rats, mice, birds and the like. For a particular subject, a BMP homolog or ortholog can be provided by the BMP nanofibers described herein (e.g., a rat BMP can be administered when the subject is a rat, a human BMP can be administered when the subject is a human, etc.).

A subject suffering from an abnormal bone condition, e.g., a fracture or microfracture, or a bone disease, e.g., osteoporosis, for example, could be treated by administering an effective amount of the BMP nanofibers described herein. As BMPs, e.g., BMP2, BMP3, BMP4, BMP5, BMP6, BMP7, BMP8, or others, direct bone mass accumulation, diseases or abnormal conditions that result from bone mass deficiency are corrected. BMP2, for example, like other bone morphogenetic proteins, plays an important role in the development of bone and cartilage. It is involved in the hedgehog pathway, TGFβ signaling pathway, and in cytokine-cytokine receptor interaction. BMP2 has been shown to stimulate the production of bone and recombinant human protein (rhBMP-2) and is available for orthopedic usage in the United States. Implantation of BMP-2 in a collagen sponge induces new bone formation and can be used for the treatment of bone defects, delayed union, and non-union (Geiger, M. et al., Adv. Drug Deliv. Rev., 55:1613-29, 2003). BMPs have also been shown to be effective in treating dental disease and abnormalities. Oral surgery and implant dentistry in particular have benefited dramatically from commercially available BMP2 (Allegrini, S. et al., J. Biomed. Mater. Res. B Appl Biomater., 68:127-31, 2004; Schlegel, K. et al., Clin. Oral Implants Res., 17: 666-72, 2006; Schliephake, H. et al., Clin. Oral Implants Res., 16:563-9, 2005).

The BMP nanofibers described herein can be administered, for example, topically, by injection, by infusion, as an implant (e.g., subcutaneously), or by other methods known in the art for locally delivering a biomaterial to the site of a bone disease or abnormality. As used herein, the term “efficacy” refers to the degree to which a desired effect is obtained. The nanofibers described herein are capable of releasing a therapeutically effective amount of an agent for treating a bone disease or abnormality. As commercial products, albeit in other formulations and forms, are available that have demonstrated effective amounts and dosages of BMPs with respect to achieving a desired effect, one of skill in the art would know how to determine a therapeutically effective amount or dose of the specific nanoparticles described herein to achieve a desired effect.

The nanofibers described herein can be administered directly, or they can be included in a treatment formulation. In addition to the BMP nanofiber, a treatment formulation can also comprise, for example, suitable excipients, preservatives, stabilizing agents, other active agents suitable for treating a disease or disorder and/or agents that increase the residence time of the nanofiber in tissues or the blood stream.

Administration of the BMP nanofibers described herein can be to a subject who, for example, has been diagnosed with a disease or condition, or with a susceptibility to a bone disease or condition, e.g., a genetic susceptibility.

The BMP nanofibers and compositions containing the BMP nanofibers described herein can be included in a kit that contains the necessary reagents for administering a BMP nanofiber or composition containing a BMP nanofiber. The kit can include, for example, components useful or necessary for the effective administration for the BMP nanofiber or composition containing the BMP nanofiber to the subject. The kit can include, for example, an applicator, sound sealing compositions, solutions for administering the BMP nanofiber or composition containing the BMP nanofiber, etc. Kits can also contain a control/reference value or a set of control/reference values indicating normal and various clinical progression stages of the disease or disorder. Kits can also contain instructions for administering the BMP nanofiber or composition containing the BMP nanofiber. The kits can also comprise, e.g., a buffering agent, a preservative, or a protein stabilizing agent. Each component of the kits can be enclosed within an individual container and all of the various containers can be within a single package.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges that can be subsequently broken down into subranges as discussed above. Finally, as will be understood by one skilled in the art, a range includes each individual member. Thus, for example, a group having one to three cells refers to groups having one, two or three cells. Similarly, a group having one to five cells refers to groups having one, two, three, four or five cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

What is claimed is:

1. A nanofiber comprising: one or more of one or more bone morphogenetic proteins, or one or more bone morphogenetic protein fragments bound to the nanofiber.

2. The nanofiber of claim 1, wherein the nanofiber is selected from the group consisting of: a carbon nanofiber, a polymeric nanofiber, an organic crystalline nanofiber, an inorganic phosphate nanofiber, a co-polymeric nanofiber, and a core-shell type nanofiber.

3. The nanofiber of claim 2, wherein the polymeric nanofiber is selected from the group consisting of: nylon, polystyrene, polyacrylonitrile, polycarbonate, poly(ethylene oxide), polyethylene terephthalate, and water soluble polymers.

4. The nanofiber of claim 1, wherein the one or more of the one or more bone morphogenetic proteins, or the one or more bone morphogenetic protein fragments, is selected from the group consisting of: BMP2; BMP3; BMP4; BMP5; BMP6; BMP7, and one or more fragments thereof.

5. The nanofiber of claim 1, wherein the one or more of the one or more bone morphogenetic proteins, or the one or more bone morphogenetic protein fragments, comprises a polypeptide with at least about 85% identity to SEQ ID NOS: 1-7 or a functional fragment thereof.

6. The nanofiber of claim 1, wherein the one or more of the one or more bone morphogenetic proteins, or the one or more bone morphogenetic protein fragments, is attached to the nanofiber by a linker molecule.

7. The nanofiber of claim 1, wherein the one or more of the one or more bone morphogenetic proteins, or the one or more bone morphogenetic protein fragments, is coupled to the nanofiber with a linkage comprising a dithio bond.

8. The nanofiber of claim 6, wherein the linker molecule is selected from the group consisting of: N-succinimidyl-3(2 pyridyldithio)-propionate, amine-containing cross-linker (SMCC), bis-maleimidohexane, dimethyl pimelimidate, dithiobis-(succinimidyl propionate), disuccinimidyl suberate, and related linkers.

9. The nanofiber of claim 6, wherein the linker comprises a hydrolysable functional group.

10. The nanofiber of claim 1, wherein the nanofiber is biodegradable.

11. The nanofiber of claim 1, wherein the nanofiber is poly(D,L-lactic lycine).

12. A nanofiber of claim 1, further comprising a pharmaceutically acceptable carrier.

13. A method of treating a bone disorder or disease in a subject, comprising: administering an effective amount of a nanofiber comprising one or more of one or more bone morphogenetic proteins, or one or more bone morphogenetic protein fragments bound to the nanofiber, wherein administration of the nanofiber results in the treatment of the bone disorder or disease.

14. The method of claim 13, wherein the bone disorder or disease is selected from the group consisting of: a fracture, a microfracture and osteoporosis.

15. The method of claim 13, wherein the treatment of the subject is achieved by an increase in the bone density of the subject.

16. A method of increasing bone density in a subject, comprising: administering an effective amount of a nanofiber comprising one or more of one or more bone morphogenetic proteins, or one or more bone morphogenetic protein fragments bound to the nanofiber, wherein administration of the nanofiber results in an increase in the bone density of the subject.

17. The method of claim 16, wherein the one or more of the one or more bone morphogenetic proteins, or the one or more bone morphogenetic protein fragments, is administered by local release from the nanofiber in a controlled manner.

18. The method of claim 17, wherein the local release of the one or more of the one or more bone morphogenetic proteins, or the one or more bone morphogenetic protein fragments is accomplished by hydrolysis of a hydrolysable functional group in a linker attaching the one or more of the one or more bone morphogenetic proteins, or the one or more bone morphogenetic protein fragments to the nanofiber.

19. The method of claim 18, wherein the hydrolysable functional group is phenolic ester or thioester.

20. A method of activating MAPK extracellular regulated kinase pathway, comprising:
administering an effective amount of a nanofiber comprising one or more of one or more bone morphogenetic proteins, or one or more bone morphogenetic protein fragments bound to the nanofiber, wherein administration of the nanofiber activates the MAPK regulated kinase pathway.

21. A method of inducing phosphorylation of SMAD1 or SMAD5 at the site of a bone disorder or disease in a subject, comprising: administering an effective amount of a nanofiber comprising one or more of one or more bone morphogenetic proteins, or one or more bone morphogenetic protein fragments bound to the nanofiber at the site of the bone disorder or disease, wherein administration of the nanofiber results in phosphorylation of SMAD1 or SMAD5 at the site of the bone disorder in the subject.

22. A kit comprising:
a) a nanofiber as in claim 1;
b) a solution suitable for storing the protein nanofiber of a); and
c) instructions for the use of the nanofiber.

23. Use of a nanofiber comprising one or more of one or more bone morphogenetic proteins, or one or more bone morphogenetic protein fragments bound to the nanofiber, for treating a bone disorder or disease.

24. The use of claim 23, wherein the bone disorder or disease is selected from the group consisting of: a fracture; a microfracture; and osteoporosis.


SEQUENCE LISTING (7 sequences)

SEQ ID NO: 1 - protein, 396 aa, Artificial Sequence
  Description of Artificial Sequence Synthetic polypeptide
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 2 - protein, 472 aa, Artificial Sequence
  Description of Artificial Sequence Synthetic polypeptide
MAGASRLLFLWLGCFCVSLAQGERPKPPFPELRKAVPGDRTAGGGPDSELQPQDKVSEHMLRLYDRYSTVQAARTPGSLEGGSQPWRPRLLREGNTVRSFRAAAAETLERKGLYIFNLTSLTKSENILSATLYFCIGELGNISLSCPVSGGCSHHAQRKHIQIDLSAWTLKFSRNQSQLLGHLSVDMAKSHRDIMSWLSKDITQLLRKAKENEEFLIGFNITSKGRQLPKRRLPFPEPYILVYANDAAISEPESVVSSLQGHRNFPTGTVPKWDSHIRAALSIERRKKRSTGVLLPLQNNELPGAEYQYKKDEVWEERKPYKTLQAQAPEKSKNKKKQRKGPHRKSQTLQFDEQTLKKARRKQWIEPRNCARRYLKVDFADIGWSEWIISPKSFDAYYCSGACQFPMPKSLKPSNHATIQSIVRAVGVVPGIPEPCCVPEKMSSLSILFFDENKNVVLKVYPNMTVESCACR

SEQ ID NO: 3 - protein, 408 aa, Artificial Sequence
  Description of Artificial Sequence Synthetic polypeptide
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 4 - protein, 454 aa, Artificial Sequence
  Description of Artificial Sequence Synthetic polypeptide
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 5 - protein, 513 aa, Artificial Sequence
  Description of Artificial Sequence Synthetic polypeptide
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 6 - protein, 431 aa, Artificial Sequence
  Description of Artificial Sequence Synthetic polypeptide
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 7 - protein, 402 aa, Artificial Sequence
  Description of Artificial Sequence Synthetic polypeptide
MTALPGPLWLLGLALCALGGGGPGLRPPPGCPQRRLGARERRDVQREILAVLGLPGRPRPRAPPAASRLPASAPLFMLDLYHAMAGDDDEDGAPAERRLGRADLVMSFVNMVERDRALGHQEPHWKEFRFDLTQIPAGEAVTAAEFRIYKVPSIHLLNRTLHVSMFQVVQEQSNRESDLFFLDLQTLRAGDEGWLVLDVTAASDCWLLKRHKDLGLRLYVETEDGHSVDPGLAGLLGQRAPRSQQPFVVTFFRASPSPIRTPRAVRPLRRRQPKKSNELPQANRLPGIFDDVHGSHGRQVCRRHELYVSFQDLGWLDWVIAPQGYSAYYCEGECSFPLDSCMNATNHAILQSLVHLMMPDAVPKACCAPTKLSATSVLYYDSSNNVILRKHRNMVVKACGCH